(19) United States
(12) Patent Application Publication
(10) Pub. No.: US 2009/0161069 A1
(43) Pub. Date: Jun. 25, 2009

(54) IRIS IMAGING LENS

(75) Inventors: Shuichi Horiguchi, Kanagawa (JP); Gentaro Irisawa, Kanagawa (JP)

Correspondence Address: PEARNE & GORDON LLP, 1801 EAST 9TH STREET, SUITE 1200, CLEVELAND, OH, 44114-3108, US

(73) Assignee: MATSUSHITA ELECTRIC INDUSTRIAL CO., LTD., Osaka (JP)

(21) Appl. No.: 12/065,402

(22) PCT Filed: Sep. 27, 2006
(86) PCT No.: PCT/JP2006/319153

§ 371 Date: Feb. 29, 2008

(30) Foreign Application Priority Data
Sep. 30, 2005 (JP) 2005-287969

(51) Int. Cl. A61B 3/10

(52) U.S. Cl. 351/213

(57) ABSTRACT

An iris imaging lens (1A) comprises a biconvex spherical lens (2A), a biconcave spherical lens (3A), and a visible light cut filter (4A), and at least one surface of the visible light cut filter (4A) is formed as a curved surface. In this configuration, the visible light cut filter (4A) serves as a lens, so that aberration is corrected not only by the biconvex spherical lens (2A) and biconcave spherical lens (3A) but also by the visible light cut filter (4A). As a result, aberration can be reduced to improve the lens performance without increasing the number of imaging lenses, and the increase in the cost of manufacturing can be limited.

### TECHNICAL FIELD

The present invention relates to an iris imaging lens to be used in an iris recognition device or the like.

### BACKGROUND ART

Conventionally, an iris recognition device that identifies an individual using iris patterns of human eyes differing from person to person has been used as a person authentication device. An iris recognition device uses infrared light to shoot a pattern of an iris. As shown in FIG. 9, an iris imaging lens 1P to be used in an iris recognition device comprises a biconvex spherical lens 2P, a meniscus-concave spherical lens 3P, and a visible light cut filter 4P, whose surfaces on both sides are parallel flat surfaces. The visible light cut filter 4P cuts unnecessary visible light and transmits infrared light. Such an iris recognition device is disclosed, for example, in Japanese Patent Laid-Open Application No. 2004-167046.

The conventional iris imaging lens 1P shown in FIG. 9, however, uses only two imaging lenses of the biconvex spherical lens 2P and the meniscus-concave spherical lens 3P. This causes a low degree of freedom in optical design, and difficulty in performing sufficient aberration correction. FIGS. 10A to 10C show spherical aberration, astigmatism, and distortion of the conventional iris imaging lens 1P. FIGS. 11A to 11H show lateral aberration in tangential and sagittal directions of the conventional iris imaging lens 1P. The conventional iris imaging lens 1P shown in FIGS. 10 and 11 has large spherical aberration and other aberration, and the lens performance is low. So, in order to improve the lens performance, aberration might be corrected by increasing the number of imaging lenses to increase a degree of freedom in optical design. In that case, however, the cost of manufacturing the iris imaging lens 1P would increase.

### DISCLOSURE OF THE INVENTION

### Problems to be Solved by the Invention

The invention has been made in the above-mentioned background. A purpose of the invention is to provide an iris imaging lens that does not require the number of imaging lenses to be increased and can limit the increase in the cost of manufacturing, and that can reduce aberration to improve the lens performance.

### Means for Solving the Problems

One aspect of the invention is an iris imaging lens, which comprises: an imaging lens; and a visible light cut filter, where at least one surface of the visible light cut filter is a curved surface.

There are other aspects of the invention as described below. This disclosure of the invention therefore intends to provide part of the aspects of the invention and does not intend to limit the scope of the invention described and claimed herein.

### BRIEF DESCRIPTION OF THE DRAWINGS

FIG. 1 shows a configuration of an iris imaging lens of a first embodiment of the invention;

FIG. 2A shows spherical aberration of the iris imaging lens of the first embodiment of the invention;

FIG. 2B shows astigmatism of the iris imaging lens of the first embodiment of the invention;

FIG. 2C shows distortion of the iris imaging lens of the first embodiment of the invention;

FIG. 3A shows lateral aberration in a tangential direction of the iris imaging lens of the first embodiment of the invention (an image height of 3.00 mm);

FIG. 3B shows lateral aberration in a sagittal direction of the iris imaging lens of the first embodiment of the invention (an image height of 3.00 mm);

FIG. 3C shows lateral aberration in a tangential direction of the iris imaging lens of the first embodiment of the invention (an image height of 2.40 mm);

FIG. 3D shows lateral aberration in a sagittal direction of the iris imaging lens of the first embodiment of the invention (an image height of 2.40 mm);

FIG. 3E shows lateral aberration in a tangential direction of the iris imaging lens of the first embodiment of the invention (an image height of 1.80 mm);

FIG. 3F shows lateral aberration in a sagittal direction of the iris imaging lens of the first embodiment of the invention (an image height of 1.80 mm);

FIG. 3G shows lateral aberration in a tangential direction of the iris imaging lens of the first embodiment of the invention (an image height of 0.00 mm);

FIG. 3H shows lateral aberration in a sagittal direction of the iris imaging lens of the first embodiment of the invention (an image height of 0.00 mm);

FIG. 4 shows a configuration of an iris imaging lens of a second embodiment of the invention;

FIG. 5A shows spherical aberration of the iris imaging lens of the second embodiment of the invention;

FIG. 5B shows astigmatism of the iris imaging lens of the second embodiment of the invention;

FIG. 5C shows distortion of the iris imaging lens of the second embodiment of the invention;

FIG. 6A shows lateral aberration in a tangential direction of the iris imaging lens of the second embodiment of the invention (an image height of 3.00 mm);

FIG. 6B shows lateral aberration in a sagittal direction of the iris imaging lens of the second embodiment of the invention (an image height of 3.00 mm);

FIG. 6C shows lateral aberration in a tangential direction of the iris imaging lens of the second embodiment of the invention (an image height of 2.40 mm);

FIG. 6D shows lateral aberration in a sagittal direction of the iris imaging lens of the second embodiment of the invention (an image height of 2.40 mm);

FIG. 6E shows lateral aberration in a tangential direction of the iris imaging lens of the second embodiment of the invention (an image height of 1.80 mm);

FIG. 6F shows lateral aberration in a sagittal direction of the iris imaging lens of the second embodiment of the invention (an image height of 1.80 mm);

FIG. 6G shows lateral aberration in a tangential direction of the iris imaging lens of the second embodiment of the invention (an image height of 0.00 mm);

FIG. 6H shows lateral aberration in a sagittal direction of the iris imaging lens of the second embodiment of the invention (an image height of 0.00 mm);

FIG. 7 shows a variation of an iris imaging lens of an embodiment of the invention;

FIG. 8 shows another variation of an iris imaging lens of an embodiment of the invention;

FIG. 9 shows a configuration of a conventional iris imaging lens;

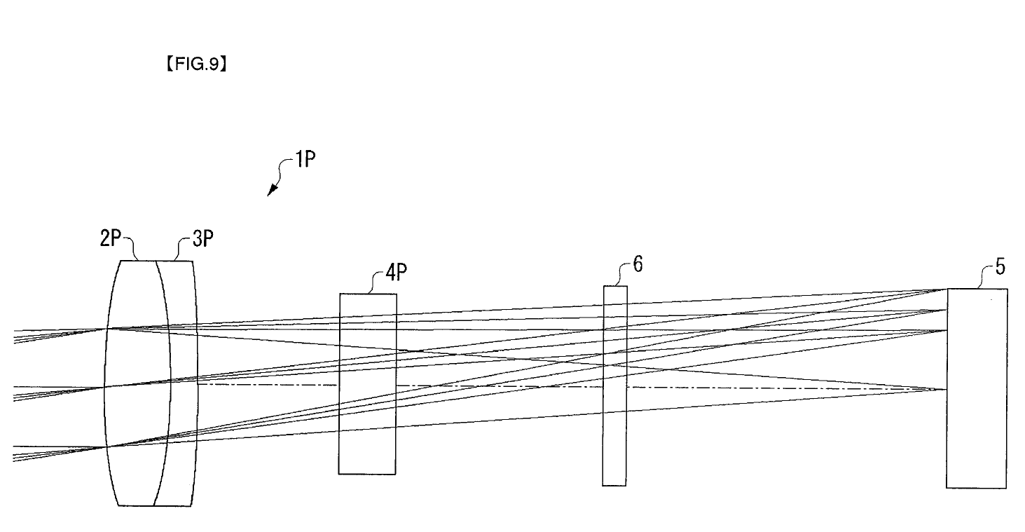

FIG. 10A shows spherical aberration of the conventional iris imaging lens;

FIG. 10B shows astigmatism of the conventional iris imaging lens;

FIG. 10C shows distortion of the conventional iris imaging lens;

FIG. 11A shows lateral aberration in a tangential direction of the conventional iris imaging lens (an image height of 3.00 mm);

FIG. 11B shows lateral aberration in a sagittal direction of the conventional iris imaging lens (an image height of 3.00 mm);

FIG. 11C shows lateral aberration in a tangential direction of the conventional iris imaging lens (an image height of 2.40 mm);

FIG. 11D shows lateral aberration in a sagittal direction of the conventional iris imaging lens (an image height of 2.40 mm);

FIG. 11E shows lateral aberration in a tangential direction of the conventional iris imaging lens (an image height of 1.80 mm);

FIG. 11F shows lateral aberration in a sagittal direction of the conventional iris imaging lens (an image height of 1.80 mm);

FIG. 11G shows lateral aberration in a tangential direction of the conventional iris imaging lens (an image height of 0.00 mm); and

FIG. 11H shows lateral aberration in a sagittal direction of the conventional iris imaging lens (an image height of 0.00 mm).

### DESCRIPTION OF THE SYMBOLS

1A and 1B: Iris imaging lens
2A: Biconvex spherical lens
2B: Meniscus-convex spherical lens
3A: Biconcave spherical lens
3B: Meniscus-concave spherical lens
4A and 4B: Visible light cut filter

### BEST MODE OF EMBODYING THE INVENTION

Now, the invention will be described in detail. However, the following detailed description and appended drawings are not intended to limit the invention. Rather, the scope of the invention is defined by the appended claims.

An iris imaging lens of the invention comprises: an imaging lens; and a visible light cut filter, where at least one surface of the visible light cut filter is a curved surface.

In this configuration, since at least one surface of the visible light cut filter is formed as a curved surface, the visible light cut filter also serves as a lens, so that the visible light cut filter, as well as the imaging lens, can correct aberration. Consequently, aberration can be reduced without increasing the number of lenses.

One surface of the visible light cut filter may be a curved surface and another surface thereof may be a flat surface.

In this configuration, since one surface of the visible light cut filter is formed as a curved surface, the visible light cut filter also serves as a lens, so that the visible light cut filter, as well, can correct aberration. In addition, since the other surface of the visible light cut filter is formed as a flat surface, the visible light cut filter can be manufactured easily, so that the cost of manufacturing can be kept low.

The curved surface of the visible light cut filter may be a rotationally-symmetric aspherical surface.

In this configuration, the visible light cut filter serves as an aspherical lens, so that the visible light cut filter can correct aberration and, in particular, can reduce spherical aberration.

A ratio of thickness of the visible light cut filter along an optical axis thereof to thickness of the visible light cut filter at a circumference of an effective radius thereof is more than or equal to 0.8 and less than or equal to 1.2.

In this configuration, a difference in transmissivity of the visible light cut filter can be prevented from occurring between light that passed through the visible light cut filter along the optical axis thereof and light that passed through the visible light cut filter at the circumference of the effective radius thereof. That is, though one surface of the visible light cut filter is a curved surface, irregularity can be prevented from occurring in spectral characteristics of the visible light cut filter. Consequently, the visible light cut filter can sufficiently serve as a filter as well as serve as a lens.

By providing a visible light cut filter whose at least one surface is a curved surface, the invention can reduce aberration to improve the lens performance without increasing the number of imaging lenses and can limit the increase in the cost of manufacturing.

Now, iris imaging lenses of embodiments of the invention will be described with reference to the drawings. These embodiments will illustrate cases of iris imaging lenses to be used in an iris recognition device or the like.

### FIRST EMBODIMENT

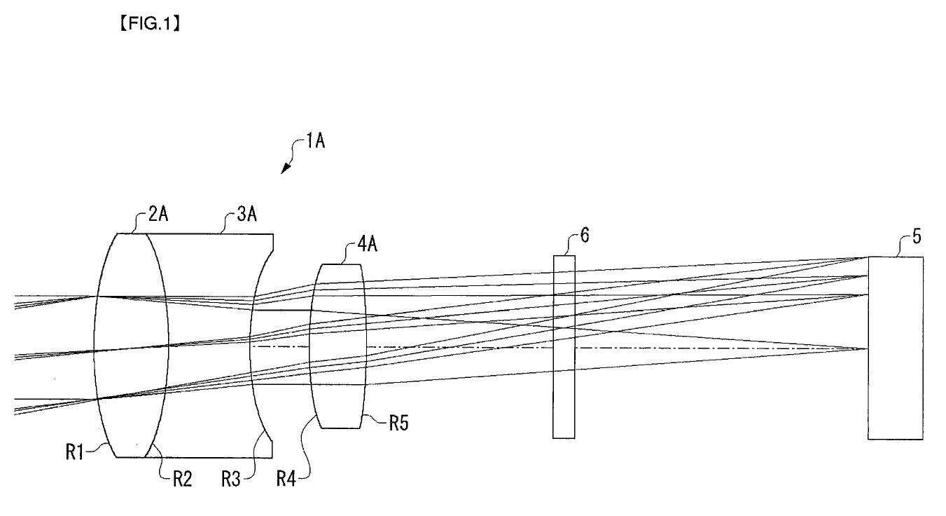

FIG. 1 shows an iris imaging lens of a first embodiment of the invention. As shown in FIG. 1, an iris imaging lens 1A comprises a biconvex spherical lens 2A made of low dispersion glass, a biconcave spherical lens 3A made of high dispersion glass, and a visible light cut filter 4A made of plastic. In this case, the biconvex spherical lens 2A and the biconcave spherical lens 3A correspond to the imaging lens. The visible light cut filter 4A is manufactured by plastic injection molding. The iris imaging lens 1A is mounted on an iris recognition device. Light condensed by the iris imaging lens 1A is converted to an imaging signal by a CCD 5 or other imaging element, and image processing is performed for iris recognition. A package glass 6 of the CCD 5 is shown in FIG. 1, but the effect of the location of the package glass 6 is negligible in terms of optical design. The ratio of a lens effective radius r1 of the biconvex spherical lens 2A to a lens spherical radius R1 thereof, r1/R1, and the ratio of a lens effective radius r2 of the biconvex spherical lens 2A to a lens spherical radius R2 thereof, r2/R2, are each set to 0.55. Preferably, these r1/R1 and r2/R2 are each set to 0.55 or lower. A refractive index n1 of the biconvex spherical lens 2A at d-line of the Fraunhofer lines is 1.569, and an Abbe's number ν1 thereof is 56.0. The radius of curvature of one lens surface of the biconvex spherical lens 2A (lens surface on the left in FIG. 1) is 7.09 mm, and the radius of curvature of the other lens surface (lens surface on the right in FIG. 1) is −6.07 mm. The thickness of the biconvex spherical lens 2A along its optical axis is 2.92 mm.

The ratio of a lens effective radius r2 of the biconcave spherical lens 3A to a lens spherical radius R2 thereof, r2/R2, and the ratio of an effective radius r3 of the biconcave spherical lens 3A to a lens spherical radius R3 thereof, r3/R3, are each set to 0.55. Preferably, these r2/R2 and r3/R3 are each set to 0.55 or lower. A refractive index n2 of the biconcave spherical lens 3A at d-line of the Fraunhofer lines is 1.620, and an Abbe's number ν2 thereof is 36.3. The radius of curvature of one lens surface of the biconcave spherical lens 3A (lens surface on the left in FIG. 1) is −6.07 mm, and the radius of curvature of the other lens surface (lens surface on the right in FIG. 1) is 5.75 mm. The thickness of the biconcave spherical lens 3A along its optical axis is 3.00 mm. As shown in FIG. 1, the biconvex spherical lens 2A and the biconcave spherical lens 3A are joined together.

The visible light cut filter 4A is manufactured by plastic molding. One surface of this visible light cut filter 4A (surface on the left in FIG. 1) is a spherical lens surface, and the other surface (surface on the right in FIG. 1) is a slightly spherical surface. A refractive index n3 of the visible light cut filter 4A at d-line of the Fraunhofer lines is 1.492, and an Abbe's number ν3 thereof is 54.67. The radius of curvature of one lens surface of the visible light cut filter 4A (lens surface on the left in FIG. 1) is 11.01 mm, and the radius of curvature of the other lens surface (lens surface on the right in FIG. 1) is −29.70 mm. The distance between the biconcave spherical lens 3A and the visible light cut filter 4A is set to 2.45 mm.

In the visible light cut filter 4A, a ratio of thickness along its optical axis to thickness at a circumference of its effective radius is set to 1.2. Specifically, the thickness of the visible light cut filter 4A along the optical axis (the central part) is 3.00 mm, and the thickness of the visible light cut filter 4A at the circumference of the effective radius (the peripheral part) is 3.60 mm. The ratio of thickness of the visible light cut filter 4A along the optical axis to thickness of the visible light cut filter 4A at the circumference of the effective radius is thus set to 1.2. In the embodiment, the visible light cut filter 4A is of a biconvex spherical lens shape (both surfaces are convex spherical surfaces). In this case, the ratio of thickness along the optical axis to thickness at the circumference of the effective radius is desirably set to more than 1.0 and less than or equal to 1.2.

The above-described biconvex spherical lens 2A, biconcave spherical lens 3A, and visible light cut filter 4A are used in the iris imaging lens 1A of the embodiment. In this iris imaging lens 1A, the focal length f is set to 25 mm, the f-number is set to 8.0, the image height is set to 3.0 mm, and the object distance is set to 320 mm.

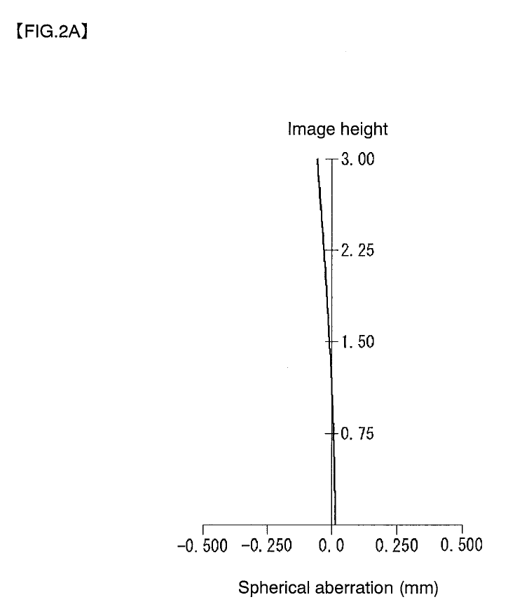

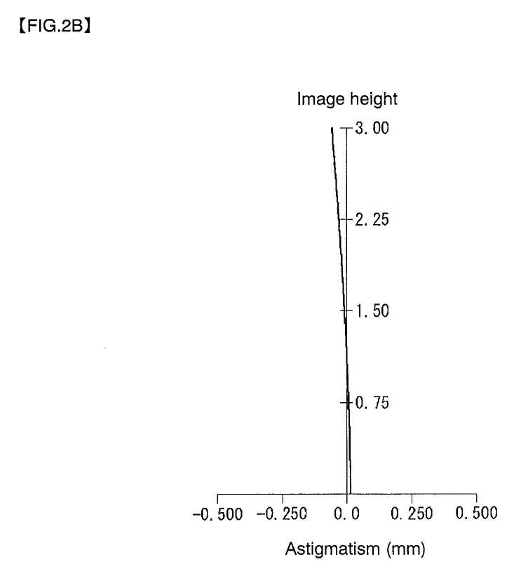

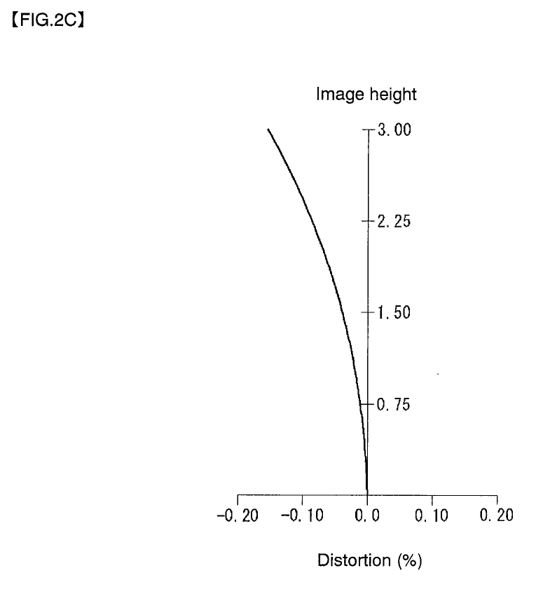

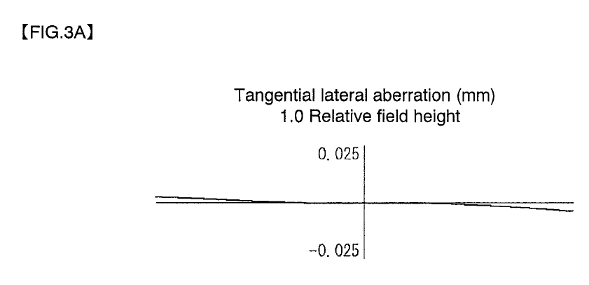

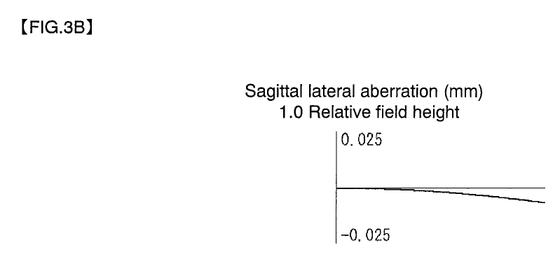

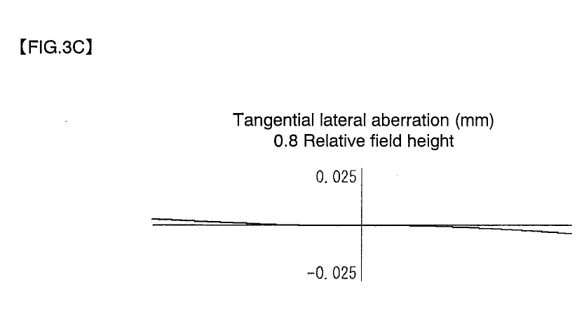

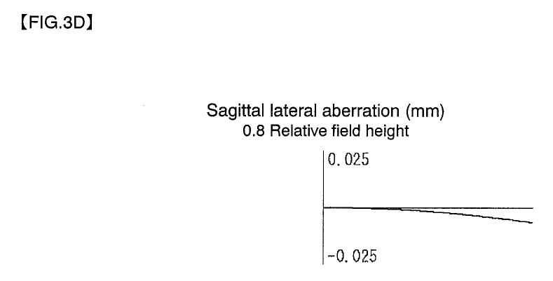

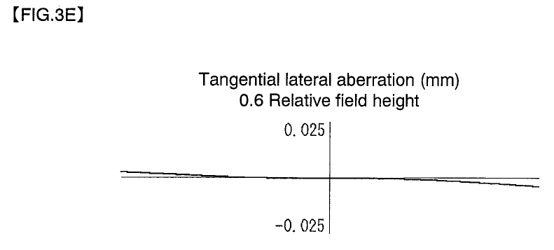

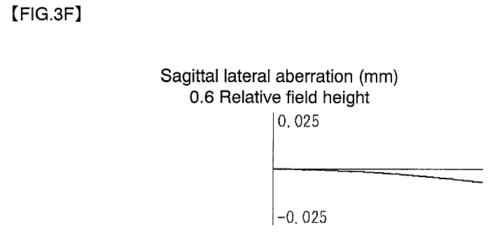

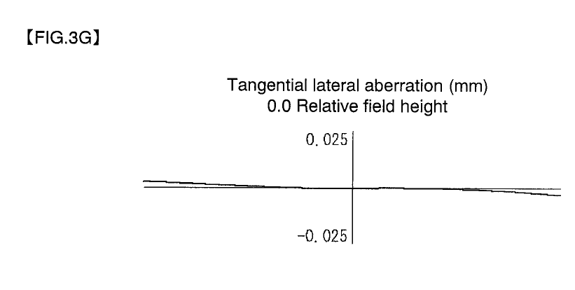

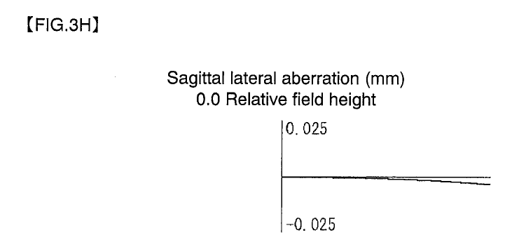

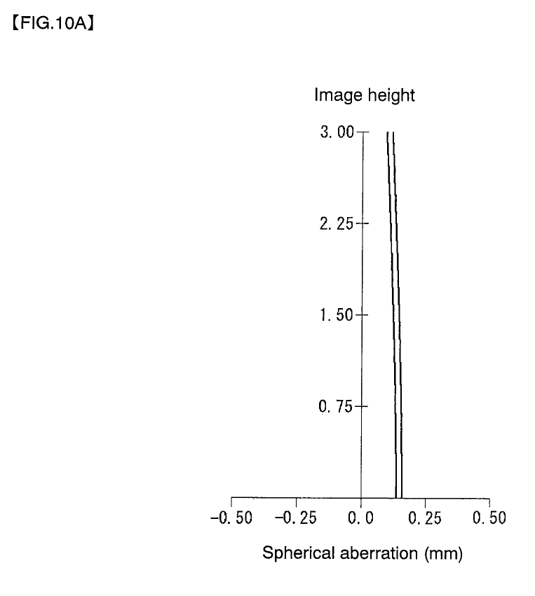

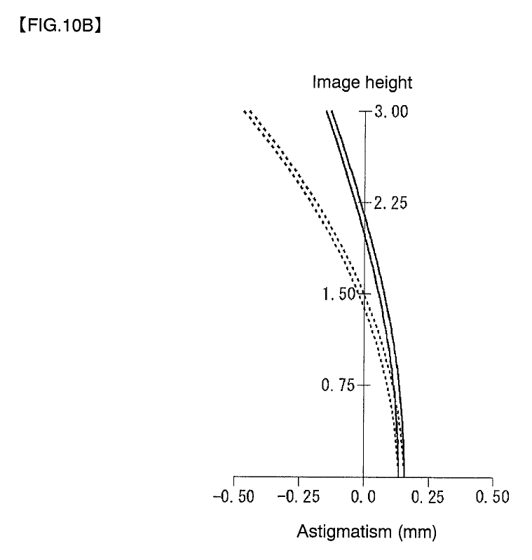

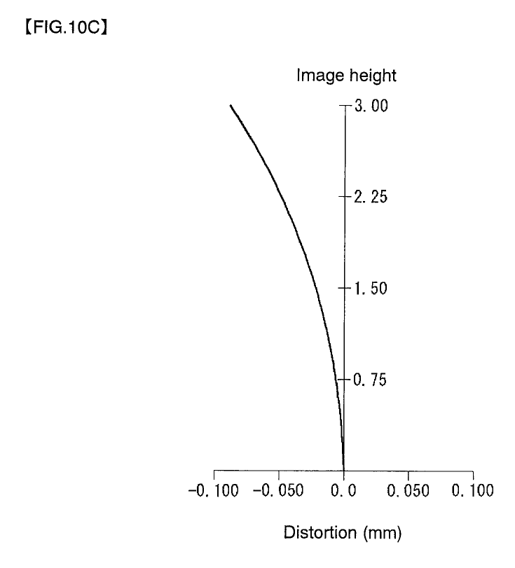

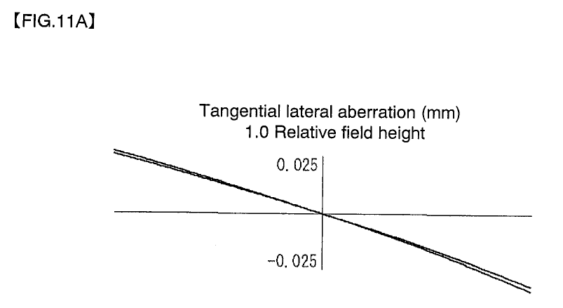

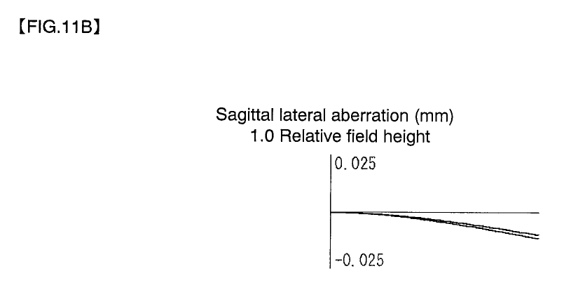

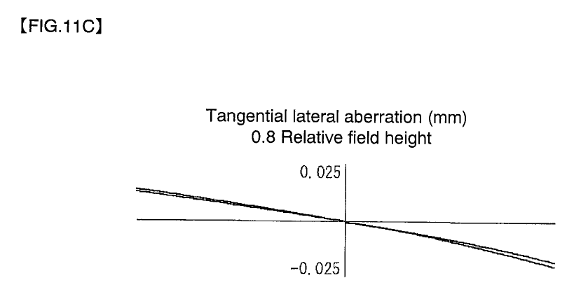

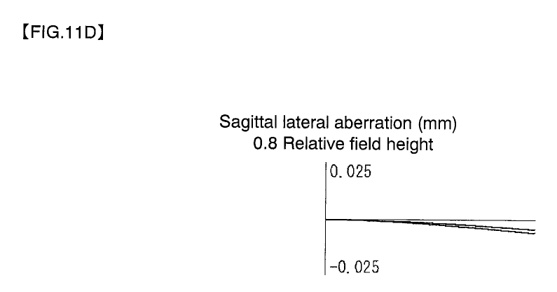

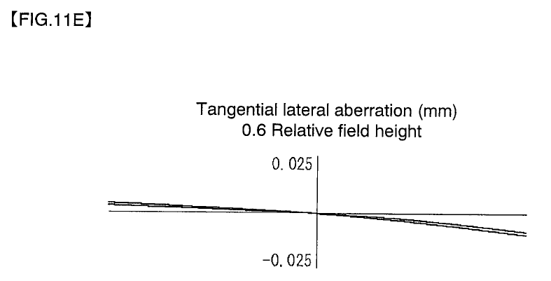

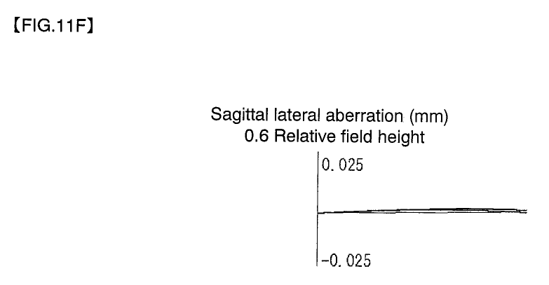

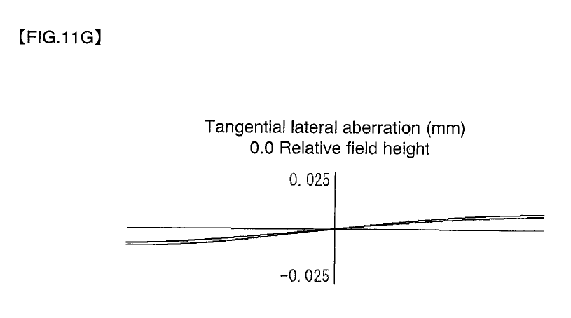

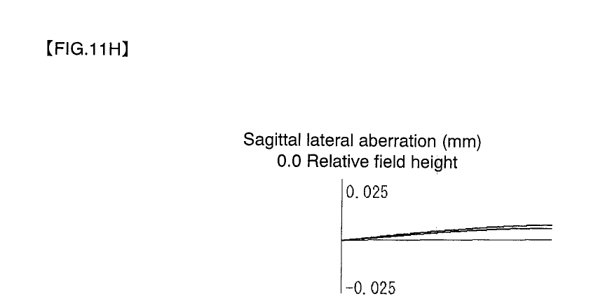

FIGS. 2 and 3 show results of calculating aberration of the iris imaging lens 1A, which is configured as above. FIGS. 2A to 2C show spherical aberration, astigmatism, and distortion of the iris imaging lens 1A of the embodiment. FIGS. 3A to 3H show lateral aberration in tangential and sagittal directions of the iris imaging lens 1A of the embodiment. As shown in FIGS. 2 and 3, it is understood that the iris imaging lens 1A of the embodiment has smaller aberration, such as spherical aberration, and an improved lens performance, as compared to the conventional iris imaging lens 1P shown in FIGS. 10 and 11.

In this iris imaging lens 1A of the first embodiment of the invention, at least one surface of the visible light cut filter 4A is a curved surface. In the above-described example, the visible light cut filter 4A, one surface of which is a curved lens surface and the other surface of which is a slightly curved lens surface, is provided. This can reduce aberration to improve the lens performance without increasing the number of imaging lenses and can limit the increase in the cost of manufacturing.

That is, in the iris imaging lens 1A of the embodiment, one surface of the visible light cut filter 4A is a curved lens surface, and the other surface is a slightly curved lens surface. Consequently, the visible light cut filter 4A serves as a lens, so that aberration can be corrected not only by the biconvex spherical lens 2A and biconcave spherical lens 3A but also by the visible light cut filter 4A. As a result, aberration can be reduced without increasing the number of imaging lenses, such as the biconvex spherical lens 2A and the biconcave spherical lens 3A.

The ratio of thickness of the visible light cut filter 4A along the optical axis to thickness of the visible light cut filter 4A at the circumference of the effective radius is set to more than 1.0 and less than or equal to 1.2, so that a difference in transmissivity of the visible light cut filter 4A can be prevented from occurring between light that passed through the visible light cut filter 4A along the optical axis (the central part) and light that passed through the visible light cut filter 4A at the circumference of the effective radius (the peripheral part). In the iris imaging lens 1A of the embodiment, since the ratio of thickness of the visible light cut filter 4A along the optical axis to thickness of the visible light cut filter 4A at the circumference of the effective radius is set to 1.2, the difference in transmissivity of the visible light cut filter 4A between along the optical axis (the central part) and at the circumference of the effective radius (the peripheral part) can be limited to six percent for light with a wavelength at which the transmissivity is 50 percent. That is, though one surface of the visible light cut filter 4A is a curved surface, irregularity can be prevented from occurring in spectral characteristics of the visible light cut filter 4A. Consequently, the visible light cut filter 4A can sufficiently serve as a filter as well as serve as a lens.

In the iris imaging lens 1A of the embodiment, the number of imaging lenses can be reduced by joining the biconvex spherical lens 2A and the biconcave spherical lens 3A together. This can reduce the amount and time of work required to manufacture and assemble the imaging lenses, so that the cost of manufacturing can be kept low. Moreover, the workability of the biconvex spherical lens 2A and biconcave spherical lens 3A can be improved by setting the ratios r1/R1, r2/R2, and r3/R3 to 0.55 or lower, the ratios being the ratios of lens effective radii to lens spherical radii of the biconvex spherical lens 2A and biconcave spherical lens 3A. Furthermore, the Abbe's number ν1 of the biconvex spherical lens 2A is set to 56 or higher and the Abbe's number ν2 of the biconcave spherical lens 3A is set to 37 or lower, so that chromatic aberration can be corrected.

### SECOND EMBODIMENT

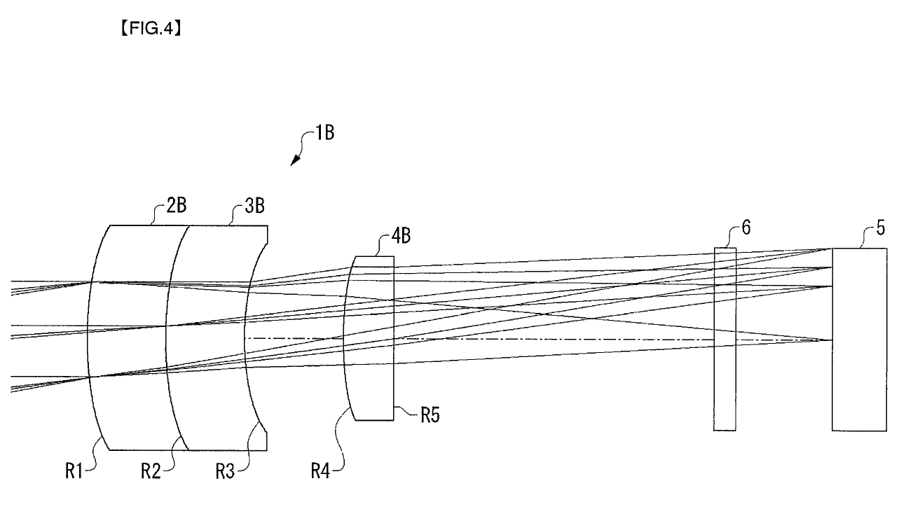

FIG. 4 shows an iris imaging lens of a second embodiment of the invention. A description will be made here of a difference in configuration of an iris imaging lens of the embodiment from that of the first embodiment shown in FIG. 1, and the same configuration as that of the first embodiment will not be mentioned in particular. In FIG. 4, an iris imaging lens 1B of the embodiment comprises a meniscus-convex spherical lens 2B made of low dispersion glass, a meniscus-concave spherical lens 3B made of high dispersion glass, and a visible light cut filter 4B made of plastic. In this case, the meniscus-convex spherical lens 2B and the meniscus-concave spherical lens 3B correspond to the imaging lens.

The ratio of a lens effective radius r1 of the meniscus-convex spherical lens 2B to a lens spherical radius R1 thereof, r1/R1, and the ratio of a lens effective radius r2 of the meniscus-convex spherical lens 2B to a lens spherical radius R2 thereof, r2/R2, are each set to 0.55. Preferably, these r1/R1 and r2/R2 are each set to 0.55 or lower. A refractive index n1 of the meniscus-convex spherical lens 2B at d-line of the Fraunhofer lines is 1.639, and an Abbe's number ν1 thereof is 55.5. The radius of curvature of one lens surface of the meniscus-convex spherical lens 2B (lens surface on the left in FIG. 4) is 9.44 mm, and the radius of curvature of the other lens surface (lens surface on the right in FIG. 4) is 24.36 mm. The thickness of the meniscus-convex spherical lens 2B along its optical axis is 3.00 mm.

The ratio of a lens effective radius r2 of the meniscus-concave spherical lens 3B to a lens spherical radius R2 thereof, r2/R2, and the ratio of an effective radius r3 of the meniscus-concave spherical lens 3B to a lens spherical radius R3 thereof, r3/R3, are each set to 0.55. Preferably, these r2/R2 and r3/R3 are each set to 0.55 or lower. A refractive index n2 of the meniscus-concave spherical lens 3B at d-line of the Fraunhofer lines is 1.487, and an Abbe's number ν2 thereof is 70.4.The radius of curvature of one lens surface of the meniscus-concave spherical lens 3B (lens surface on the left in FIG. 4) is 24.36 mm, and the radius of curvature of the other lens surface (lens surface on the right in FIG. 4) is 8.59 mm. The thickness of the meniscus-concave spherical lens 3B along its optical axis is 3.00 mm. As shown in FIG. 4, the meniscus-convex spherical lens 2B and the meniscus-concave spherical lens 3B are joined together.

The visible light cut filter 4B is manufactured by plastic molding. One surface of this visible light cut filter 4B (surface on the left in FIG. 4) is a rotationally-symmetric aspherical lens surface, and the other surface (surface on the right in FIG. 4) is a flat surface. An aspherical radius R5 of the visible light cut filter 4B is 15.67. A refractive index n3 of the visible light cut filter 4B at d-line of the Fraunhofer lines is 1.492, and an Abbe's number ν3 thereof is 54.67.The thickness of the visible light cut filter 4B along its optical axis is set to 2.00 mm, which is 1.15 times as long as the thickness of the visible light cut filter 4B at a circumference of its effective radius. That is, the ratio of thickness of the visible light cut filter 4B along the optical axis to thickness of the visible light cut filter 4B at the circumference of the effective radius is set to 1.15. The aspherical surface of the visible light cut filter 4B is defined by the following aspherical surface definitional equation:

Z=[ch²/{1+(1−c²h²)^{-0.5}}]+Ah⁴+Bh⁶+Ch⁸+Dh¹⁰

c=1/r

where A, B, C, and D are constants, and are set in the embodiment as: A=−0.1195×10⁻³, B=−0.4512×10⁻⁵, C=0, and D=0.

The above-described visible light cut filter 4B is used in the iris imaging lens 1B of the embodiment. In this iris imaging lens 1B, the focal length f is set to 25 mm, the f-number is set to 8.0, the image height is set to 3.0 mm, and the object distance is set to 320 mm.

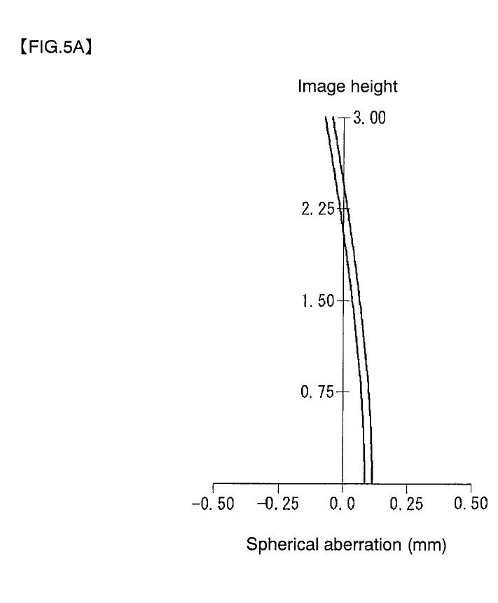

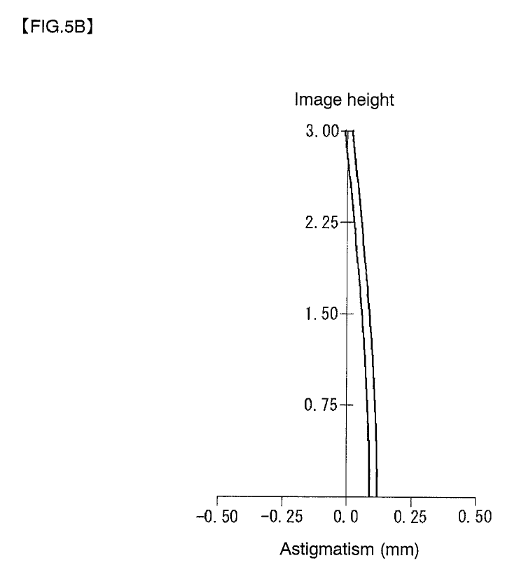

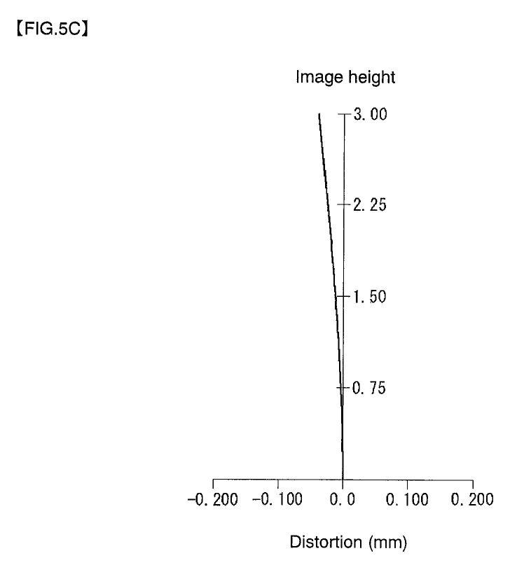

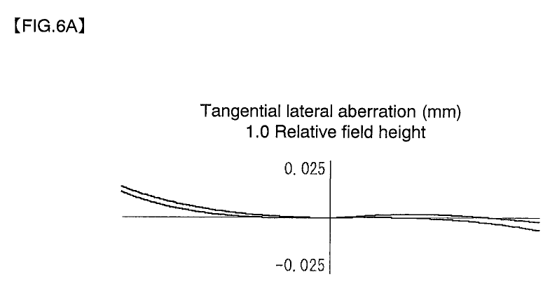

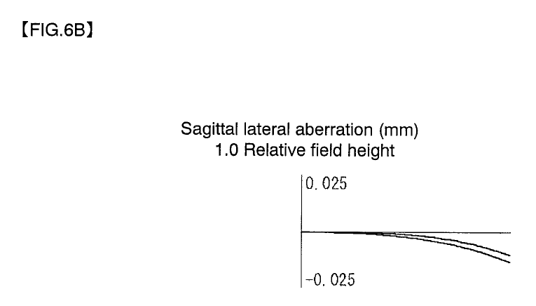

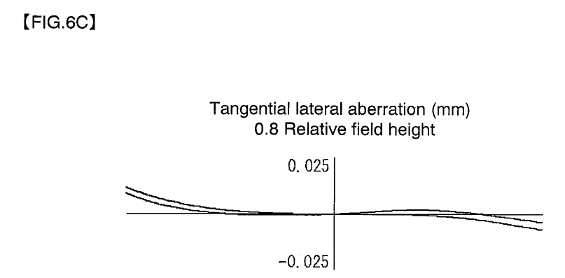

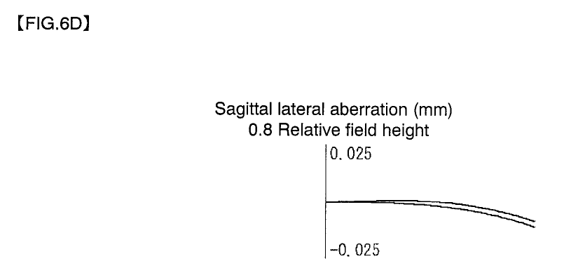

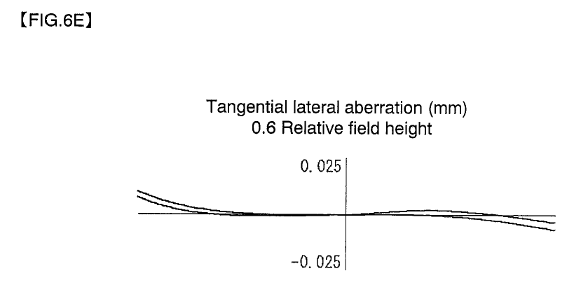

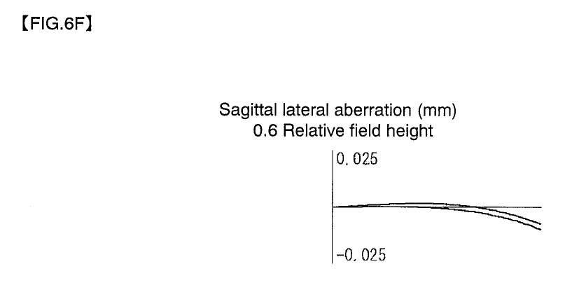

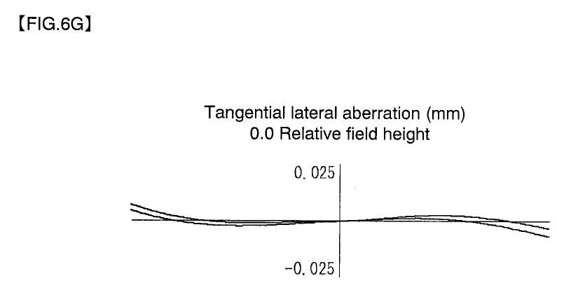

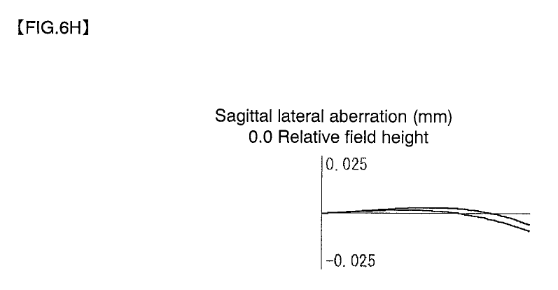

FIGS. 5 and 6 show results of calculating aberration of the iris imaging lens 1B, which is configured as above. FIGS. 5A to 5C show spherical aberration, astigmatism, and distortion of the iris imaging lens 1B of the embodiment. FIGS. 6A to 6H show lateral aberration in tangential and sagittal directions of the iris imaging lens 1B of the embodiment. As shown in FIGS. 5 and 6, it is understood that the iris imaging lens 1B of the embodiment has smaller aberration, such as spherical aberration, and an improved lens performance, as compared to the conventional iris imaging lens 1P shown in FIGS. 10 and 11.

Provided with the visible light cut filter 4B, one surface of which is a curved lens surface and the other surface of which is a flat surface, this iris imaging lens 1B of the second embodiment of the invention can reduce aberration to improve the lens performance without increasing the number of imaging lenses and can limit the increase in the cost of manufacturing.

That is, in the iris imaging lens 1B of the embodiment, one surface of the visible light cut filter 4B is a curved lens surface, and the other surface is a flat surface. Consequently, the visible light cut filter 4B serves as a lens, so that aberration can be corrected not only by the meniscus-convex spherical lens 2B and meniscus-concave spherical lens 3B but also by the visible light cut filter 4B. In addition, since the other surface of the visible light cut filter 4B is a flat surface, a mold to be used for manufacturing the visible light cut filter 4B can be manufactured at a low cost, so that the cost of manufacturing can be kept low.

In the iris imaging lens 1B of the embodiment, since the curved surface of the visible light cut filter 4B is a rotationally-symmetric aspherical surface, the visible light cut filter 4B serves as an aspherical lens, so that the visible light cut filter 4B can correct aberration and, in particular, can reduce spherical aberration. As with the previous embodiment, the ratio of thickness of the visible light cut filter 4B along the optical axis to thickness of the visible light cut filter 4B at the circumference of the effective radius is set to more than 1.0 and less than or equal to 1.2, so that a difference in transmissivity of the visible light cut filter 4B can be prevented from occurring between light that passed through the visible light cut filter 4B along the optical axis (the central part) and light that passed through the visible light cut filter 4B at the circumference of the effective radius (the peripheral part). In the iris imaging lens 1B of the embodiment, since the ratio of thickness of the visible light cut filter 4B along the optical axis to thickness of the visible light cut filter 4B at the circumference of the effective radius is set to 1.15, the difference in transmissivity of the visible light cut filter 4B between along the optical axis (the central part) and at the circumference of the effective radius (the peripheral part) can be limited to five percent for light with a wavelength at which the transmissivity is 50 percent. That is, though one surface of the visible light cut filter 4B is a curved surface, irregularity can be prevented from occurring in spectral characteristics of the visible light cut filter 4B. Consequently, the visible light cut filter 4B can sufficiently serve as a filter as well as serve as a lens.

Also in the iris imaging lens 1B of the embodiment, the number of imaging lenses can be reduced by joining the meniscus-convex spherical lens 2B and the meniscus-concave spherical lens 3B together. This can reduce the amount and time of work required to manufacture and assemble the imaging lenses, so that the cost of manufacturing can be kept low. Moreover, the workability of the meniscus-convex spherical lens 2B and meniscus-concave spherical lens 3B can be improved by setting the ratios r1/R1, r2/R2, and r3/R3 to 0.55 or lower, the ratios being the ratios of lens effective radii to lens spherical radii of the meniscus-convex spherical lens 2B and meniscus-concave spherical lens 3B. Furthermore, the Abbe's number ν1 of the meniscus-convex spherical lens 2B is set to 55 or higher and the Abbe's number ν2 of the meniscus-concave spherical lens 3B is set to 71 or lower, so that chromatic aberration can be corrected.

While there have been described embodiments of the invention with reference to illustrations, the scope of the invention is not limited thereto, and modifications and variations may be made thereto within the claimed scope according to purposes.

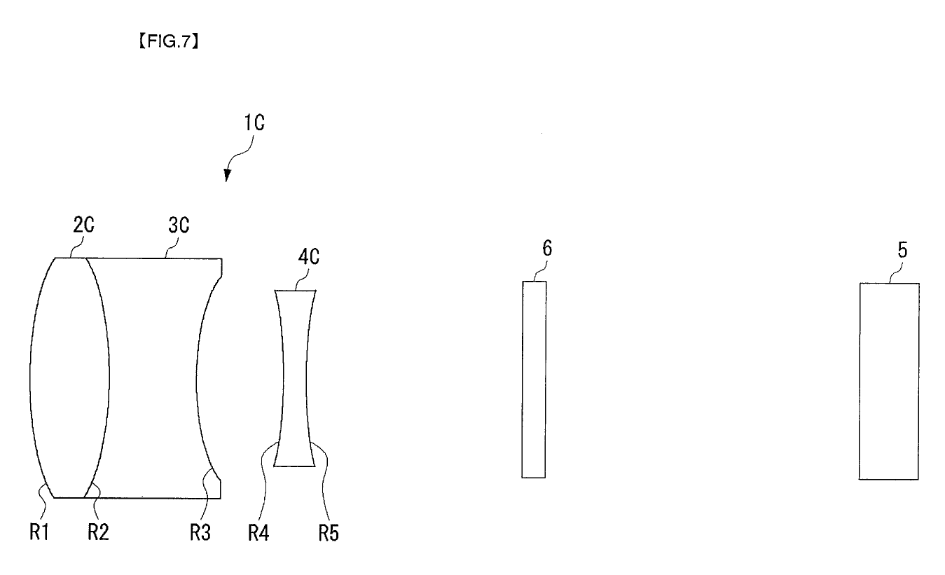

Besides the above-described embodiments, a visible light cut filter 4C may be of a biconcave spherical lens shape (both surfaces may be concave spherical surfaces) as shown in FIG. 7. In this case, the ratio of thickness along the optical axis to thickness at the circumference of the effective radius is desirably set to more than or equal to 0.8 and less than 1.0. In the example of FIG. 7, for example, the ratio of thickness along an optical axis to thickness of the visible light cut filter 4C at the circumference of the effective radius is set to 0.8.

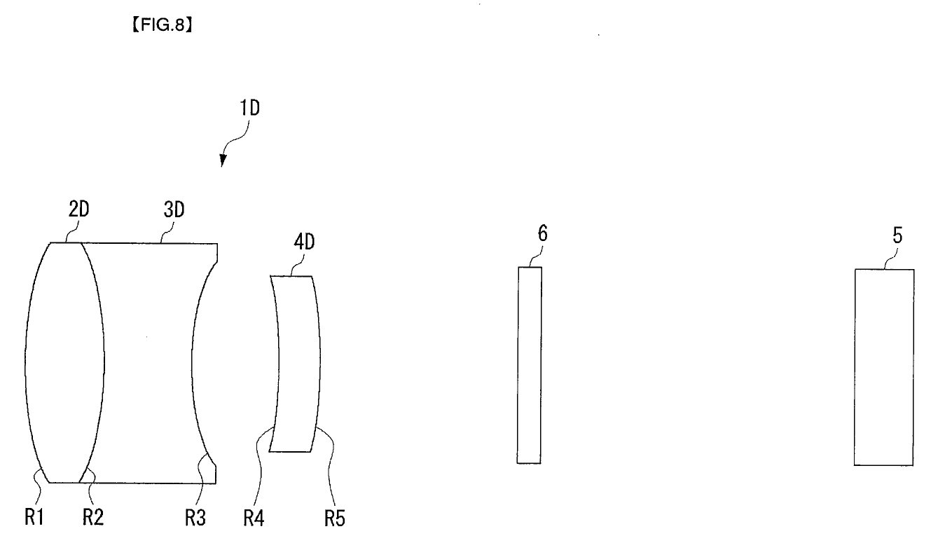

As shown in FIG. 8, one surface of a visible light cut filter 4D may be a convex spherical surface, and the other surface may be a concave spherical surface. In this case, the ratio of thickness along the optical axis to thickness at the circumference of the effective radius is desirably set to more than or equal to 0.8 and less than or equal to 1.2. In the example of FIG. 8, for example, the ratio of thickness along an optical axis to thickness of the visible light cut filter 4D at the circumference of the effective radius is set to 1.0.

While there have been described what are at present considered to be preferred embodiments of the invention, it will be understood that various modifications and variations may be made thereto, and it is intended that appended claims cover all such modifications and variations as fall within the true spirit and scope of the invention.

### INDUSTRIAL APPLICABILITY

As above, the iris imaging lens of the invention has advantages of being able to reduce aberration to improve the lens performance without increasing the number of imaging lenses and of being able to limit the increase in the cost of manufacturing, and is useful as an iris imaging lens or the like to be used in an iris recognition device or the like.

1. An iris imaging lens comprising:
an imaging lens; and
a visible light cut filter,
wherein at least one surface of the visible light cut filter is a curved surface.

2. The iris imaging lens according to claim 1, wherein one surface of the visible light cut filter is a curved surface and another surface thereof is a flat surface.

3. The iris imaging lens according to claim 1, wherein the curved surface of the visible light cut filter is a rotationally-symmetric aspherical surface.

4. The iris imaging lens according to claim 1, wherein a ratio of thickness of the visible light cut filter along an optical axis thereof to thickness of the visible light cut filter at a circumference of an effective radius thereof is more than or equal to 0.8 and less than or equal to 1.2.
